(19) United States
(12) Patent Application Publication
(10) Pub. No.: US 2023/0380748 A1
(43) Pub. Date: Nov. 30, 2023

(54) ENCODING HEART RATE IN A BEAT MARKER DISPLAY

(71) Applicant: Preventice Solutions, Inc., Rochester, MN (US)

(72) Inventors: David Robert Engebretsen, Cannon Falls, MN (US); Benjamin Adam Teplitzky, Lakeville, MN (US)

(21) Appl. No.: 18/201,334

(22) Filed: May 24, 2023

Related U.S. Application Data
(60) Provisional application No. 63/345,973, filed on May 26, 2022.

(51) Int. Cl. A61B 5/352; A61B 5/0245

(52) U.S. Cl. CPC A61B 5/352; A61B 5/0245; A61B 5/339

(57) ABSTRACT

A method includes identifying, using a machine learning model, beats and locations of peaks of the beats within electrocardiogram data. The method further includes determining heart rate over time based, at least in part, on the beats and the locations of the peaks. The method further includes displaying the heart rate over time and the locations of the peaks in single annotations on a graph.

### CROSS REFERENCE TO RELATED APPLICATION

This application claims priority to Provisional Application No. 63/345,973, filed May 26, 2022, which is herein incorporated by reference in its entirety.

### TECHNICAL FIELD

The present disclosure relates to devices, methods, and systems for analyzing cardiac activity and cardiac events.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Cardiac monitoring can be used, for example, to identify abnormal cardiac rhythms, so that critical alerts can be provided to patients, physicians, or other care providers and patients can be treated.

### SUMMARY

In Example 1, a method includes identifying, using a machine learning model, beats and locations of peaks of the beats within electrocardiogram (ECG) data. The method further includes determining heart rate over time based, at least in part, on the beats and the locations of the peaks. The method further includes displaying the heart rate over time and the locations of the peaks in single annotations on a graph.

In Example 2, the method of Example 1, wherein a distance between the locations of the peaks on the graph is based on time elapsed between two successive R-waves within the ECG data.

In Example 3, the method of Example 2, wherein the distance is based on peaks of the R-waves.

In Example 4, the method of any of the preceding Examples, wherein a height of each of the single annotations is based on the heart rate over time.

In Example 5, the method of Example 4, wherein the height of each of the single annotations relative to each other is proportional to the heart rate over time associated with each of the single annotations.

In Example 6, the method of any of the preceding Examples, wherein the determining the heart rate over time is determined by machine learning model.

In Example 7, the method of any of the preceding Examples, wherein the heart rate over time is based on a timing between a successive pair of beats.

In Example 8, the method of any of the preceding Examples, wherein the single annotations are displayed chronologically.

In Example 9, the method of any of the preceding Examples, further including displaying the ECG data on the graph.

In Example 10, the method of Example 9, wherein the single annotations are superimposed on the ECG data on the graph.

In Example 11, the method of either of Example 9 or Example 10, wherein the locations of the peaks coincide with peaks in the ECG data on the graph.

In Example 12, the method of any of Examples 9-11, wherein a height of the single annotation is greater than a height of peaks within the ECG data.

In Example 13, a computer program product comprising instructions to cause one or more processors to carry out the steps of the method of Examples 1-12.

In Example 14, a computer-readable medium having stored thereon the computer program product of Example 13.

In Example 15, a computer comprising the computer-readable medium of Example 14.

In Example 16, a system includes a server with: a database, a first processor, and a first computer-readable medium having a first set of computer-executable instructions embodied thereon. The first set of instructions are configured to be executed by the first processor to cause the first processor to: identify, using a machine learning model, beats and locations of peaks of the beats within ECG data; determine heart rate over time based, at least in part, on the beats and the locations of the peaks; and generate a second set of instructions to cause a remote computer to display the heart rate over time and the locations of the peaks in single annotations on a graph.

In Example 17, the system of Example 16, wherein the heart rate over time is determined by the machine learning model.

In Example 18, the system of Example 16, wherein the heart rate over time is based on a timing between a successive pair of beats.

In Example 19, the system of Example 18, wherein the timing is converted to beats per minute.

In Example 20, the system of Example 16, wherein the first set of instructions are configured to be executed by the first processor to cause the first processor to: combine the ECG data and the second set of instructions into a package of data for transmission to the remote computer.

In Example 21, the system of Example 16, further including the remote computing system with: a user interface (UI), a second processor, and a second computer-readable medium having the second set of computer-executable instructions embodied thereon. The second set of instructions are configured to be executed by the second processor to cause second first processor to: after receiving a package of data comprising the ECG data and the second set of instructions: display the heart rate over time and the locations of the peaks in single annotations on the graph on the UI.

In Example 22, the system of Example 21, wherein a height of each of the single annotations is based on the heart rate over time.

In Example 23, the system of Example 21, wherein the ECG data is displayed on the graph.

In Example 24, the system of Example 23, wherein the single annotations are superimposed on the ECG data on the graph.

In Example 25, a method includes identifying, using a machine learning model, beats and locations of peaks of the beats within ECG data. The method further includes determining heart rate over time based, at least in part, on the beats and the locations of the peaks. The method further includes causing the heart rate over time and the locations of the peaks to be displayed in single annotations on a graph.

In Example 26, the method of Example 25, wherein a height of each of the single annotations is based on the heart rate over time.

In Example 27, the method of Example 25, wherein the single annotations are superimposed on the ECG data on the graph.

In Example 28, the method of Example 27, wherein the locations of the peaks coincide with peaks in the ECG data on the graph.

In Example 29, the method of Example 25, wherein the heart rate over time is based on a timing between a successive pair of beats.

In Example 30, the method of Example 29, wherein the timing is converted to beats per minute.

In Example 31, a method includes receiving a package of ECG data and metadata associated with the ECG data. The ECG data includes beats with respective locations of peaks. The metadata includes determined heart rate overtime based, at least in part, on the beats and the locations of the peaks. The method further includes displaying the heart rate over time and the locations of the peaks in single annotations on a graph.

In Example 32, the method of Example 31, wherein a height of each of the single annotations is based on the heart rate over time.

In Example 33, the method of Example 31, further including displaying the ECG on the graph.

In Example 34, the method of Example 33, wherein the single annotations are superimposed on the ECG data on the graph.

In Example 35, the method of Example 33, wherein the single annotations coincide with peaks in the ECG data on the graph.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, remote computer, and user interface, in accordance with certain instances of the present disclosure.

FIG. 3 shows a view of a report building page, in accordance with certain instances of the present disclosure.

FIGS. 4-7 show different plots of heart data, in accordance with certain instances of the present disclosure.

FIG. 8 shows a flow diagram depicting an illustrative method for creating and displaying annotations of heart rate over time and peak locations, in accordance with instances of the disclosure.

FIG. 9 shows a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

The present disclosure relates to devices, methods, and systems for facilitating analysis of cardiac activity and cardiac events (e.g., abnormal cardiac rhythms or other issues).

Electrocardiogram (ECG) data of a patient can be used to monitor cardiac activity and/or identify whether the patient has experienced a cardiac event. One type of cardiac activity measurement is referred to as heart rate over time or HRt. Trends or patterns of a patient's heart rate over time can suggest certain cardiac activity or events.

Typically, in ECG analysis tools, a patient's heart rate over time is displayed simply using text. For example, if a patient's heart rate over time for a given period of time was 150 beats per minute, the display may numerically show “150” as part or adjacent to a plot of associated ECG data. However, it can be challenging to identify patterns or trends with just numerical text. Certain instances of the present disclosure are accordingly directed to systems, methods, and devices for encoding heart rate information in plots of ECG data via annotations.

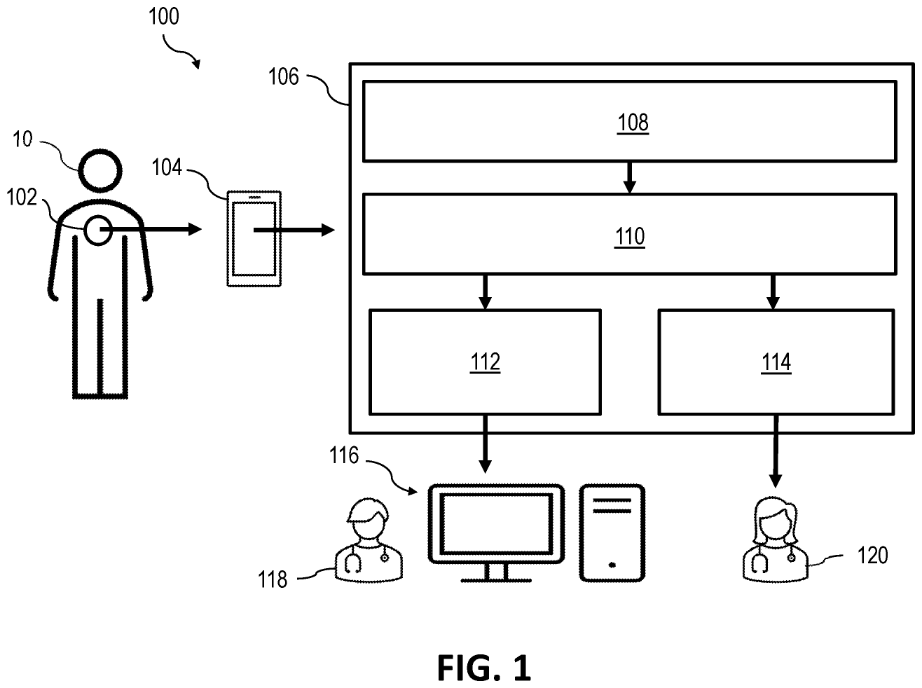

FIG. 1 illustrates a patient 10 and an example system 100. The system 100 includes a monitor 102 attached to the patient 10 to detect cardiac activity of the patient 10. The monitor 102 may produce electric signals that represent the cardiac activity in the patient 10. For example, the monitor 102 may detect the patient's heart beating (e.g., using infrared sensors, electrodes) and convert the detected heartbeat into electric signals representing ECG data. The monitor 102 communicates the ECG data to a mobile device 104 (e.g., a mobile phone).

The mobile device 104 may include a program (e.g., mobile phone application) that receives, processes, and analyzes the ECG data. For example, the program may analyze the ECG data and detect or flag cardiac events (e.g., periods of irregular cardiac activity) contained within the ECG data. Because ECG data may be getting continuously generated, the amount of ECG data can be overwhelming to store and process locally on the mobile device 104. As such, the mobile device 104 can periodically transmit chunks of the ECG data to another device or system, which can process, append together, and archive the chunks of the ECG data and metadata (e.g., time, duration, detected/flagged cardiac events) associated with the chunks of ECG data. In certain instances, the monitor 102 may be programmed to transmit the ECG data directly to the other device or system without utilizing the mobile device 104. Also, in certain instances, the monitor 102 and/or the mobile device 104 includes a button or touch-screen icon that allows the patient 10 to initiate an event. Such an indication can be recorded and communicated to the other device or system. In other instances involving multi-day studies, the ECG data and associated metadata are transmitted in larger chunks.

### Cardiac Event Server

In the example shown in FIG. 1, the mobile device 104 transmits the ECG data (and associated metadata, if any) to a cardiac event server 106 (hereinafter “the server 106” for brevity). The server 106 includes multiple platforms, layers, or modules that work together to process and analyze the ECG data such that cardiac events can be detected, filtered, prioritized, and ultimately reported to a patient's physician for analysis and treatment. In the example of FIG. 1, the server 106 includes one or more machine learning models 108 (e.g., deep neural networks), a cardiac event router 110, a report platform 112, and a notification platform 114. Although only one server 106 is shown in FIG. 1, the server 106 can include multiple separate physical servers, and the various platforms/modules/layers can be distributed among the multiple servers. Each of the platforms/modules/layers can represent separate programs, applications, and/or blocks of code where the output of one of the platforms/modules/layers is an input to another of the platforms/modules/layers. Each platform/module/layer can use application programming interfaces (APIs) to communicate between or among the other platforms/modules/layers as well as systems and devices external to the server 106.

The server 106 applies the machine learning model 108 to the ECG data to determine various cardiac data and events. As examples, the machine learning model 108 may identify heart beats contained in the ECG data and may determine information about heart rates over time.

In certain embodiments, the machine learning model 108 includes two paths, where the first path is a deep convolutional neural network and the second path is a deep fully-connected neural network. The deep convolutional neural network receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The deep fully connected neural network receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

If the machine learning model 108 determines that the ECG data most closely resembles a labeled ECG data associated with a cardiac event, then the machine learning model 108 may determine that the patient 10 has experienced that cardiac event. Additionally, the machine learning model 108 may measure or determine certain characteristics of the cardiac activity of the patient 10 based on the ECG data. For example, the machine learning model 108 may determine a heart rate, a duration, or a beat count of the patient 10 during the cardiac event based on the ECG data. The machine learning model 108 may also determine a confidence level for each classification or identification of a cardiac event. The confidence level is an indication of certainty or uncertainty in the accuracy of the machine learning model's classification or identification. The server 106 stores the cardiac event (and associated metadata such as information like heart rate, duration, beat count, etc.) in a database for storage. Subsequently, the server 106 may retrieve the cardiac event and associated information from the database.

In certain instances, the mobile device 104 (or monitor 102) may initially classify a cardiac event based on the ECG data. The server 106 may then re-classify or confirm the cardiac event using the machine learning model 108. Doing so allows for a more computationally-expensive analysis of the ECG data to be performed using the computing resources of the server 106, rather than the limited resources of the mobile device 104.

In certain instances, once the ECG data is processed by the machine learning model 108, the ECG data is made available for the report platform 112. As will be described in more detail below, the report platform 112 can be accessed by a remote computer 116 (e.g., client device such as a laptop, mobile phone, desktop computer, and the like) by a user at a clinic or lab 118.

In other instances, the cardiac event router 110 is used to determine what platform further processes the ECG data based on the classification associated with the cardiac event. For example, if the identified cardiac event is severe, the cardiac event router 110 can flag or send the ECG data, etc., to the notification platform 114. The notification platform 114 can be programmed to send notifications (along with relevant ECG data and associated metadata) immediately to the patient's physician/care group remote computer 116 and/or to the patient 10 (e.g., to their computer system, e-mail, mobile phone application).

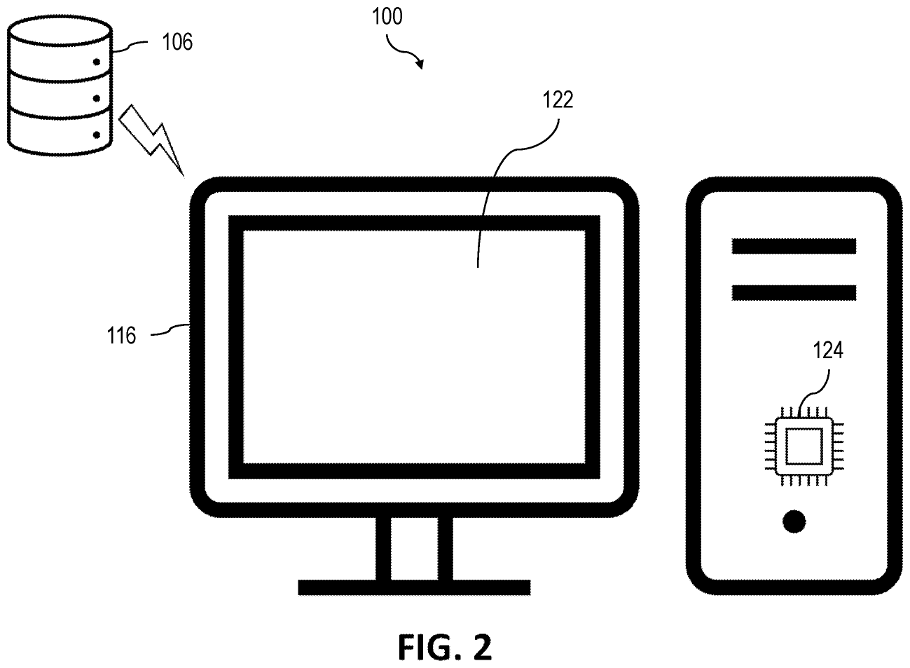

FIG. 2 shows the server 106 communicatively coupled (e.g., via a network) to the remote computer 116. In the example of FIG. 2, the remote computer 116 includes a monitor showing a user interface 122 (hereinafter “the UI 122” for brevity) that displays features of the report platform 112 hosted by the server 106. The UI 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the UI 122 to log into the report platform 112 via a web browser such that the user can use and interact with the report platform 112. When the user at the clinic or lab 118 is ready to analyze ECG data of a patient, the user can select a patient's profile through the UI 122.

The server 106 (e.g., via programming associated with the report platform 112) can start a process for sending data to the remote computer 116. This data includes the ECG data and metadata associated with the ECG data. As noted above, once the ECG data from the monitored patients has been collected, the machine learning model 108 may determine certain characteristics of the cardiac activity of the patient 10 based on the ECG data, including estimating that a cardiac event has occurred and associating or generating metadata for the determined events. The metadata can include information about the patient 10, a heart rate of the patient 10 during the cardiac event, a duration of the cardiac event, a beat count of the cardiac event, a confidence level of the machine learning model's identification of the cardiac event, and/or a beat classification (e.g., normal, ventricular, supraventricular, unclassified). In certain embodiments, the machine learning model 108 assigns each beat with a beat classification and also assigns, for certain groups and patterns of beats, a cardiac event type (e.g., atrial fibrillation, ventricular tachycardia, flutter). To distinguish among the beats, each individual beat can therefore be assigned a unique identifier (e.g., a unique number).

Accessing, processing, and displaying one or more days' worth of ECG data and metadata can consume a large amount of computing resources, network bandwidth resources, and human resources. To help alleviate burdens on these resources, the server 106 (e.g., via the report platform 112) can selectively transmit packages of ECG data and metadata to the remote computer 116.

The initial packages of data can include: (1) short strips (e.g., 60-second strips) of ECG data surrounding detected cardiac events, (2) metadata associated with the strips, and (3) executable code (e.g., JavaScript code). In certain instances, only the ECG data associated with highest priority cardiac events are initially transferred. After the initial packages of data are transmitted from the server to the remote computer 116, additional packages of data can be transmitted in response to selections made by the user in the UI 122.

### Report Build Page

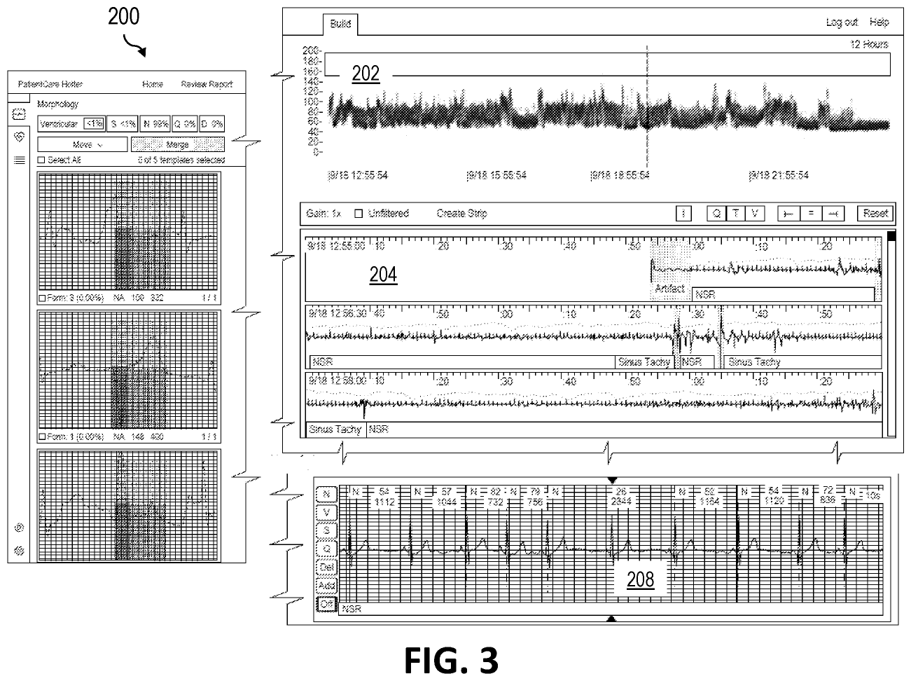

With the initial packages of data received at the remote computer 116, the user has access (via the UI 122) to a report build page 200 shown in FIG. 3. This page is generated at the remote computer 116 based on the packages of data and is selectively displayed via the UI 122. The report build page 200 facilitates analysis of cardiac events.

FIG. 3 shows a screenshot of the report build page 200, which is used by a user to review and analyze a patient's ECG data and metadata. The report build page 200 includes multiple windows for displaying data, plots, icons, links, markers, indicators, and the like.

Window 202 displays a heart rate plot of multiple days' worth of ECG data. This window 202 provides an initial visual insight into which periods of time appear to contain abnormal heart rate activity. In the example of FIG. 3, the window 202 displays four days of ECG data, although shorter or longer time periods could be displayed by default or changed by the user.

Window 204 allows the user to view a shorter plot of ECG data. For example, the window 204 may display ECG data associated with a detected cardiac event along with ECG data preceding and following the detected event. This window 204 provides visual insight into the onset of a detected event and whether the detected event is perhaps an artifact, follow-on event, etc.

Window 208 shows a plot of ECG data (e.g., approximately 10 beats) that is shorter than the plots of windows 202 and 204. Window 208 displays a closer-up view of a portion of the ECG data of windows 202 and 204. The user can use window 204 to select which shorter set of ECG data is displayed in the window 208. Each of the windows 202, 204, and 208 can include markers, indicators, icons, etc., to visually note the location of detected cardiac events within the strips of ECG data.

### Heart Rate Over Time and Peak Location Encoded in Single Annotation

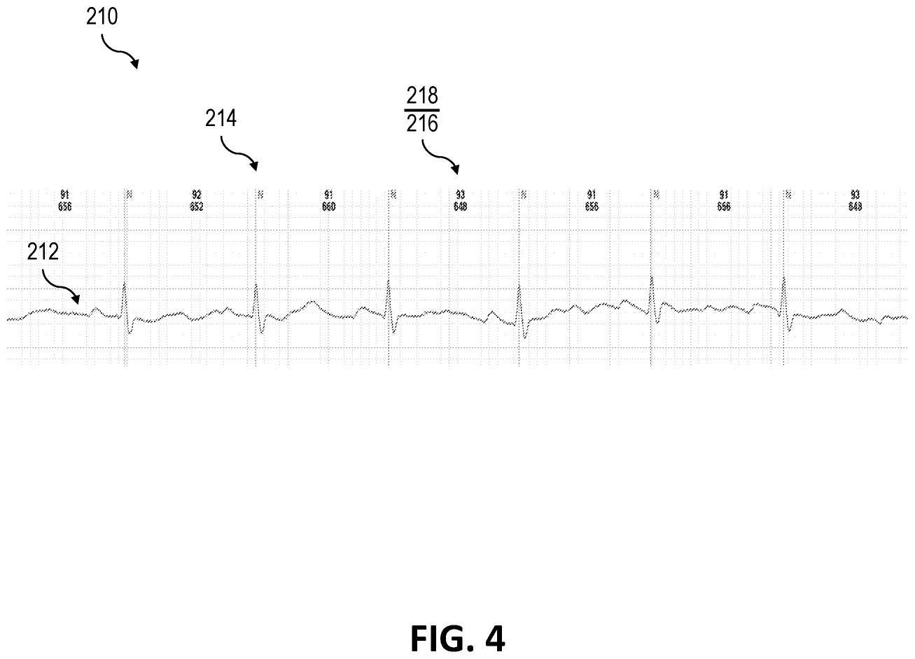

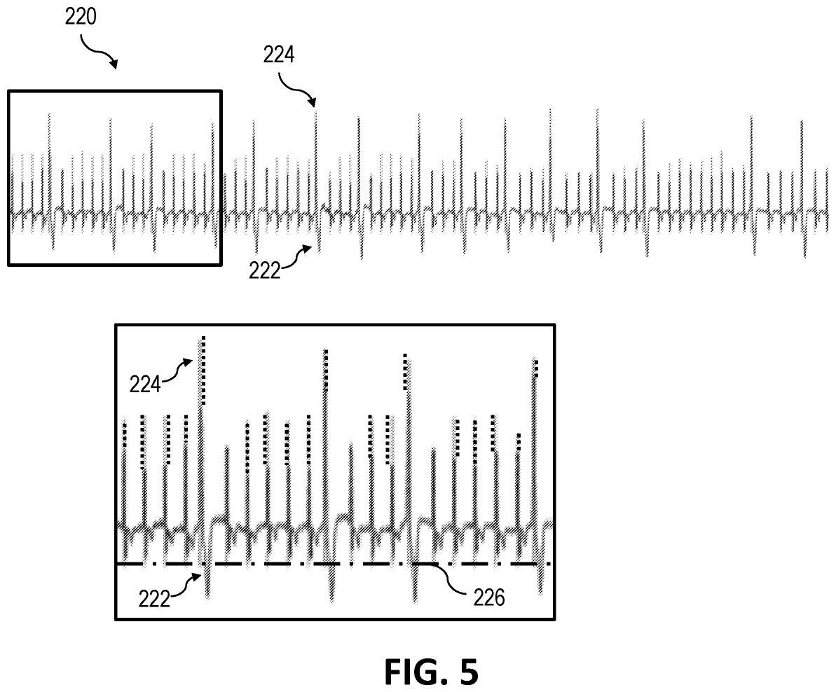

As noted above, one type of cardiac activity measurement is referred to as heart rate over time, which can suggest certain cardiac activity or events based on trends or patterns. FIG. 4 shows a graph of ECG data that displays heart rate over time without encoded annotations (e.g., displayed simply as numerals in text). In contrast, FIGS. 5-7 show graphs of ECG data with encoded annotations. These graphs can be displayed on the UI 122 at the remote computer 116, for example, in one of the windows of the report build page 200. As described in more detail below, the peak location and heart rate over time is encoded within a single annotation.

FIG. 4 shows a graph 210 with a plot 212 of ECG data. In this example, the ECG data includes six beats. Each beat is labeled with a beat classification 214, which is shown as “N” for the beats in FIG. 4, indicating normal beats. Between each pair of beats at the top of the graph 210 is an RR interval value 216 and a heart rate over time value 218. The bottom value is the RR interval 216, which indicates the time elapsed (in milliseconds) between two successive R-waves of QRS portions of ECG data. Put another way, RR intervals measure the time between two peaks of successive R-waves. The upper value is the heart rate over time value 218 in terms of beats per minute. In the example of FIG. 4, the heart rate over time is displayed simply using text (e.g., 91, 92, 91, 93, 91, and so on). However, it can be challenging to identify patterns or trends in the heart rate over time with just text.

FIG. 5 shows a graph 220 with a plot 222 of ECG data and annotations 224. The plot 222 of ECG data is shown by a line (e.g., a continuous line) in a darker shade, and the annotations 224 are shown by vertical lines in a lighter shade. In the lower zoomed-in image of FIG. 5, the annotations 224 are further identified with dashed lines. Although the annotations 224 are shown as lines in FIG. 5, other types of annotations such as bars can be used and displayed on the graph 220.

Each annotation 224 represents a patient's heart rate over time. In certain instances, the heart rate over time is the instantaneous heart rate, meaning that the heart rate is based on the most recent successive pair of beats. This heart rate calculation can be measured in milliseconds and converted into a heart rate in terms of beats per minute. In certain instances, the heart rate overtime is determined by calculating how many beats have occurred in the last 60 seconds of ECG data.

As noted above, the machine learning model 108 of FIG. 1 initially identifies the beats and locations of the peaks (e.g., the locations of the peaks in time) within the ECG data. Based on the identified beats and the locations of the peaks, the machine learning model 108 can determine the heart rate overtime. Instead of—or in addition to—displaying the determined heart rate over time using text on the graph 220, the heart rate over time is shown visually using the annotations 224.

Each determined heart rate over time can be displayed in a separate single annotation 224 on the graph 220. A height of each annotation 224 can indicate the heart rate over time. For example, an annotation 224 that is taller than another annotation 224 indicates a higher heart rate over time (e.g., a higher number of beats per minute). The relative heights of the annotations 224 can be proportional to the determined heart rate over time. For example, an annotation 224 can be displayed as being twice as tall (relative to another annotation) when the annotation 224 represents a heart rate over time that has twice as many beats per minute compared to the other heart rate over time. In certain instances, the graph 220 includes numerical indicators along the Y-axis of the graph 220 that show how a given height translates to the number of beats per minute.

Because the locations of the annotations 224 indicate the timing of the peaks, the distance between the annotations 224 on the graph 220 can represent the time elapsed between two successive R-waves within the ECG data (e.g., the distance between peaks of the R-waves within a QRS portion of the ECG data).

As shown in FIG. 5, the plot 222 of ECG data and the annotations 224 are displayed on the same graph 220 chronologically, and the plot 222 and the annotations are superimposed on each other. Because the location of each annotation 224 indicates the location of the associated peak, the peaks within the ECG data coincide with or are aligned with the annotations 224. As such, the graph 220 can include an annotation 224 for each beat. In certain instances, if the machine learning model 108 does not initially identify a beat correctly, then no annotation will be created and displayed.

As shown in FIG. 5, in certain instances, the height of the annotations 224 is higher than the height of the peak of the associated ECG data. The base of each annotation 224 can start at the same vertical level (e.g., along phantom line 226 in FIG. 5), whereas the depth and shape of the troughs of the plot 222 of the ECG data vary based on the actual measured data.

Each annotation 224 can be aligned with the locations of the peaks of the beats. Therefore, each single annotation 224 can indicate the determined heart rate over time as well as the peak location. As such, the peak location and the heart rate over time is encoded within a single annotation.

By displaying the peak location and the heart rate over time in a single annotation, a technician reviewing the graph 220 can efficiently see patterns or trends of the heart rate over time. For example, when ECG data is displayed in 1 to 5 minutes intervals, the single annotation visually provides information for a technician to interpret the represented data/values and detect patterns or trends. More specifically, heart rate over time order, disorder, and patterns can easily be viewed and used to differentiate rhythms such as normal sinus, atrial fibrillation, and sinus arrythmia. In the example of FIG. 5, premature beats can be identified by the taller annotations 224. Moreover, representing the heart rate over time and peak location in a single annotation allows the user interface to be simpler and number of indicators on the interface reduced.

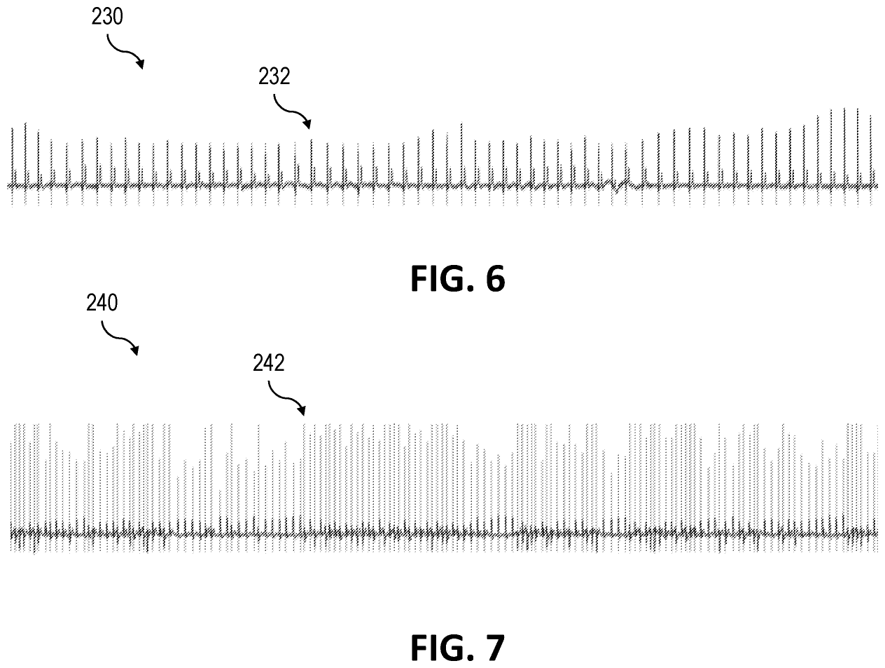

FIG. 6 shows another graph 230 with annotations 232—each of which represents a heart rate over time and a peak location. In the example of FIG. 6, the height of the annotations 232 shows a trend where a patient's heart rate over time changes smoothly over time. Such a trend indicates a sinus arrythmia event.

FIG. 7 shows another graph 240 with annotations 242—each of which represents a heart rate over time and a peak location. In the example of FIG. 7, the height of the annotations 242 shows a trend where a patient's heart rate over time varies chaotically over time. Such a trend indicates an atrial fibrillation event.

### Methods

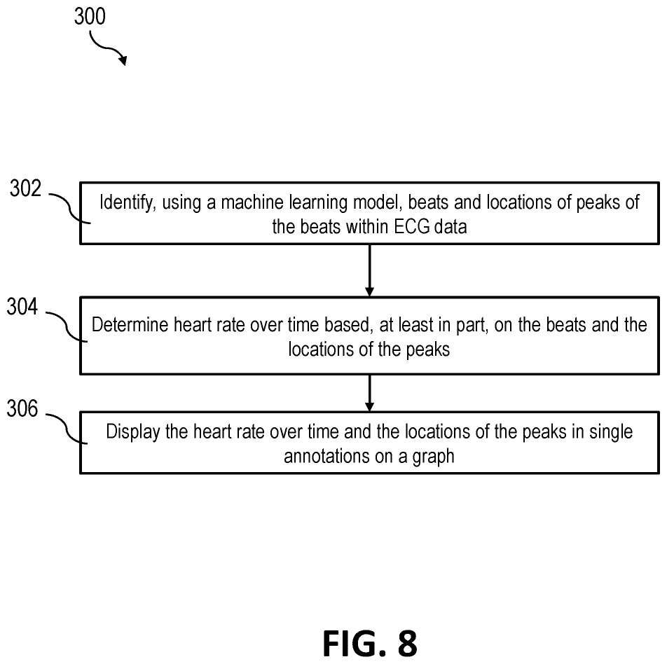

FIG. 8 shows a block diagram of a method 300 for creating and displaying annotations of heart rate over time and peak locations. The method 300 includes identifying, using a machine learning model, beats and locations of peaks of the beats within ECG data (block 302 in FIG. 8). The method 300 further includes determining heart rate over time based, at least in part, on the beats and the locations of the peaks (block 304 in FIG. 8). The method 300 further includes displaying the heart rate over time and the locations of the peaks in single annotations on a graph (block 306 in FIG. 8).

### Computing Devices and Systems

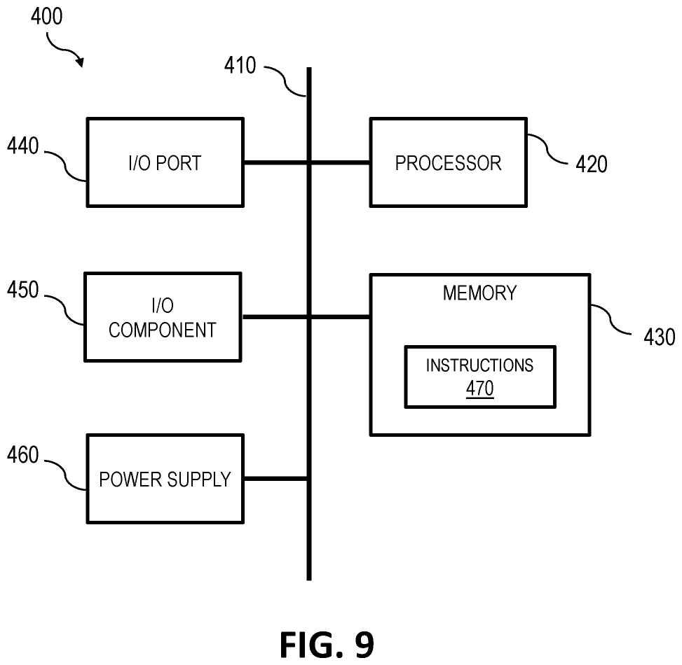

FIG. 9 is a block diagram depicting an illustrative computing device 400, in accordance with instances of the disclosure. The computing device 400 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 5 and described below. For example, the mobile device 104, the server 106, and the remote computer 116 can each include their own set of components shown in FIG. 5 and described below.

In instances, the computing device 400 includes a bus 410 that, directly and/or indirectly, couples one or more of the following devices: a processor 420, a memory 430, an input/output (I/O) port 440, an I/O component 450, and a power supply 460. Any number of additional components, different components, and/or combinations of components may also be included in the computing device 400.

The bus 410 represents what may be one or more busses (such as, for example, an address bus, data bus, or combination thereof). Similarly, in instances, the computing device 400 may include a number of processors 420, a number of memory components 430, a number of I/O ports 440, a number of I/O components 450, and/or a number of power supplies 460. Additionally, any number of these components, or combinations thereof, may be distributed and/or duplicated across a number of computing devices.

In instances, the memory 430 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical media; magnetic cassettes, magnetic tape, magnetic disk storage or other magnetic storage devices; data transmissions; and/or any other medium that can be used to store information and can be accessed by a computing device. In instances, the memory 430 stores computer-executable instructions 470 for causing the processor 420 to implement aspects of instances of components discussed herein and/or to perform aspects of instances of methods and procedures discussed herein. The memory 430 can comprise a non-transitory computer readable medium storing the computer-executable instructions 470.

The computer-executable instructions 470 may include, for example, computer code, machine-useable instructions, and the like such as, for example, program components capable of being executed by one or more processors 420 (e.g., microprocessors) associated with the computing device 400. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 470 may be configured to be executed by the processor 420 and, upon execution, to cause the processor 420 to perform certain processes. In certain instances, the processor 420, memory 430, and instructions 470 are part of a controller such as an application specific integrated circuit (ASIC), field-programmable gate array (FPGA), and/or the like. Such devices can be used to carry out the functions and steps described herein.

The I/O component 450 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. A system comprising:
a server comprising: a database, a first set of one or more processors, and a first computer-readable medium having a first set of computer-executable instructions embodied thereon, the first set of instructions configured to be executed by the first set of one or more processors to cause the server to:
identify, using a machine learning model, beats and locations of peaks of the beats within electrocardiogram (ECG) data,
determine heart rate over time based, at least in part, on the beats and the locations of the peaks, and
generate a second set of instructions to cause a remote computer to display the heart rate over time and the locations of the peaks in single annotations on a graph.

2. The system of claim 1, wherein the heart rate over time is determined by the machine learning model.

3. The system of claim 1, wherein the heart rate over time is based on a timing between a successive pair of beats.

4. The system of claim 3, wherein the timing is converted to beats per minute.

5. The system of claim 1, wherein the first set of instructions are configured to be executed by the first set of one or more processors to cause the server to: combine the ECG data and the second set of instructions into a package of data for transmission to the remote computer.

6. The system of claim 1, further comprising:
the remote computing system comprising: a user interface (UI), a second set of one or more processors, and a second computer-readable medium having the second set of computer-executable instructions embodied thereon, the second set of instructions configured to be executed by the second set of one or more processors to cause the remote computing system to:
after receiving a package of data comprising the ECG data and the second set of instructions: display the heart rate over time and the locations of the peaks in single annotations on the graph on the UI.

7. The system of claim 6, wherein a height of each of the single annotations is based on the heart rate over time.

8. The system of claim 6, wherein the ECG data is displayed on the graph.

9. The system of claim 8, wherein the single annotations are superimposed on the ECG data on the graph.

10. A method comprising:
identifying, using a machine learning model, beats and locations of peaks of the beats within electrocardiogram (ECG) data;
determining heart rate over time based, at least in part, on the beats and the locations of the peaks; and
causing the heart rate over time and the locations of the peaks to be displayed in single annotations on a graph.

11. The method of claim 10, wherein a height of each of the single annotations is based on the heart rate over time.

12. The method of claim 10, wherein the single annotations are superimposed on the ECG data on the graph.

13. The method of claim 12, wherein the locations of the peaks coincide with peaks in the ECG data on the graph.

14. The method of claim 10, wherein the heart rate over time is based on a timing between a successive pair of beats.

15. The method of claim 14, wherein the timing is converted to beats per minute.

16. A method comprising:
receiving a package of electrocardiogram (ECG) data and metadata associated with the ECG data, the ECG data including beats with respective locations of peaks, the metadata including determined heart rate over time based, at least in part, on the beats and the locations of the peaks; and
displaying the heart rate over time and the locations of the peaks in single annotations on a graph.

17. The method of claim 16, wherein a height of each of the single annotations is based on the heart rate over time.

18. The method of claim 16, further comprising:
displaying the ECG data on the graph.

19. The method of claim 18, wherein the single annotations are superimposed on the ECG data on the graph.

20. The method of claim 18, wherein the single annotations coincide with peaks in the ECG data on the graph.
